(19) United States
(12) Patent Application Publication
(10) Pub. No.: US 2008/0161784 A1
(43) Pub. Date: Jul. 3, 2008

(54) Method and system for remotely controlled MR-guided focused ultrasound ablation

(76) Inventors: Joseph M. Hogan, Oconomowoc, WI (US); Giora Sat, Waukesha, WI (US); David M. Goldhaber, Shorewood, WI (US)

Correspondence Address: GE HEALTHCARE;c/o FLETCHER YODER, PC, P.O. BOX 692289, HOUSTON, TX, 77269-2289, US

(21) Appl. No.: 11/588,043

(22) Filed: Oct. 26, 2006

(51) Int. Cl. A61B 18/04; A61B 5/05

(52) U.S. Cl. 606/27; 600/410

(57) ABSTRACT

A remotely controlled magnetic resonance guided focused ultrasound system includes a magnetic resonance scanner and focused ultrasound transducer assembly at a location where a patient is to be treated, and a control station which is not immediately at the location where the patient is to be treated. The control station may be remote from the patient location, and connected to the scanner and the transducer assembly via a network. A local controller aids in acquiring magnetic resonance guide images and thermographic images during sonication. The sonication itself is controlled remotely by a surgeon by the control station.

### BACKGROUND

The present invention relates generally to the field of non-interventional surgical techniques, and more particularly to a magnetic resonance guided focused ultrasound ablation technique that can be remotely controlled by a physician working in conjunction with a clinician or a clinical team local to a patient.

A number of surgical procedures have been developed and are presently in use wherein images are used to assist in guiding a surgeon in removing, ablating or otherwise treating tissues. Most such techniques are based upon acquisition of images before the surgical procedure, although an increasing number call for acquisition and processing of images during the actual surgical procedure. Many of the procedures developed to-date are interventional in nature, including catheterization procedures, cardiac ablation procedures, stint positioning and vascular surgical procedures, exploratory procedures, and so forth. Certain procedures, however, do not necessarily require actual intervention in the conventional sense. One such procedure, is commonly referred to as magnetic resonance-guided focused ultrasound (MRgFUS).

In MRgFUS procedures, guide images are typically acquired and focused ultrasonic pulses are created to heat tissue at a particular location within a patient for destruction of the tissue. Tissue destruction may occur at various temperatures, such as typically on the order of 160° F., although the actual tissue destruction is generally a result of overall heating in a region over a period of time. By modulating the pulses applied to an ultrasonic transducer, then, a surgeon can carefully direct destruction of tissues within the patient. During the time that ultrasonic pulses are delivered to the patient, magnetic resonance thermographic or heat images are made to aid in localizing the tissue ablation, and to track increase in temperature, indicative of heat accumulation and tissue destruction. Such procedures have proven extremely effective for certain types of treatment, such as for ablation of uterine fibroids. The process may be used to provide other treatments, and, more generally, the focused beam of ultrasonic energy penetrates soft tissue and produces well-defined regions of protein denaturation, irreversible cell damage, and coagulative necrosis at specific target locations.

In performing MRgFUS procedures, a trained physician or surgeon is required. The surgeon works in conjunction with a surgical or clinical team that aids in producing the guide and thermographic images. However, current applications of MRgFUS are believed to be limited in present technologies by the requirement that the physician be physically present at the location where the patient is treated. That is, current technologies highly integrate the control of the focused ultrasound (FUS) energy delivery subsystems with the imaging operations used to guide and to provide feedback regarding thermal load of the tissues. Similar tight integration is the norm for all systems that control the operation of either or both of these subsystems, effectively requiring the patient, the clinical team and the surgeon to be co-located during MRgFUS procedures.

There is a need, therefore, for improvements in MRgFUS procedures. There is, at present, a particular need for a technique that would allow more such procedures to be provided by specialists without requiring the specialists to travel to a patient location, or the patient to travel to a specialized facility where the surgeon or other specialist is located.

### BRIEF DESCRIPTION

The present invention provides a novel approach to MRgFUS surgical procedures designed to respond to such needs. The invention may be provided in a wide range of settings, and allows the surgeon to perform the procedure without being located immediately at the location of the patient or vise versa. The technique is based upon separation of the functions, and of certain of the systems used in delivering MRgFUS treatment. In accordance with certain aspects of the invention, an MRgFUS system or suite is provided at a patient location, with the mechanisms for delivering focused ultrasound energy to the patient. The MRgFUS system further includes the magnetic resonance imaging component that allows for generation of guide images and thermographic images. However, the control components need not be located at the patient location. That is, the images, control signals, and feedback are transmitted between the MRgFUS system and a control station where the surgeon can oversee and control the delivery of FUS energy to the patient. The control station and the MRgFUS system may be coupled to one another by a network, which may include wide area networks, such as the Internet.

The resulting system provides for a complete paradigm shift in the manner in which MRgFUS surgery can be performed. That is, a surgeon may perform one or many such surgeries at one or many remote locations from a single control station. The MRgFUS systems may be, therefore, many times multiplied and located at widely dispersed geographic locations, including at locations which are difficult to access or where specialists capable of performing the procedures are generally not available.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical overview of a remotely controlled MRgFUS system in accordance with aspects of the present technique;

FIG. 2 is a more detailed diagrammatical overview of certain of the functional components which may be included in the system of FIG. 1;

FIG. 3 is a flow chart illustrating exemplary control logic for performing a remotely-controlled MRgFUS procedure; and

FIG. 4 is a diagrammatical overview of an extended remotely-controlled MRgFUS system including a number of surgical suites for performing operations under the control of a single control station.

### DETAILED DESCRIPTION

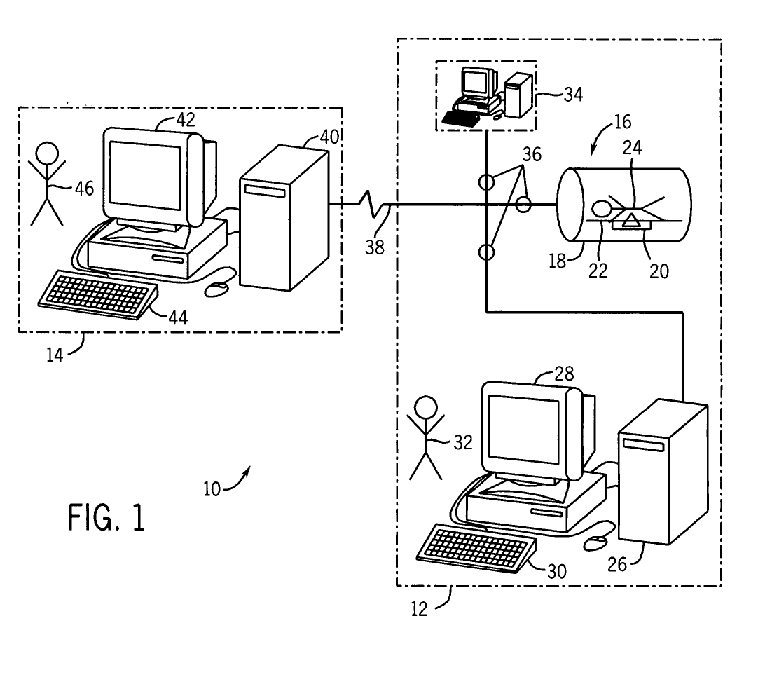

Referring to the drawings, and first to FIG. 1, a remotely-controlled MRgFUS system 10 is illustrated as including an MRgFUS system or suite 12 and a remote surgical control workstation 14. In general, the MRgFUS system may be similar to those available in the field, and will include a magnetic resonance scanner and FUS station 16. As will be appreciated by those skilled in the art, the station 16 includes a magnetic resonance scanner 18 designed to produce magnetic resonance images of a patient. The scanner may be configured for other procedures than the FUS procedure described herein, and may be any suitable form of scanner. The station 16 includes an FUS transducer assembly 20 positioned below a table 22 within a patient bore in the scanner. The table may be advanced into the patient bore and a patient 24 is positioned on the table for the MRgFUS procedure described below.

As will be appreciated by those skilled in the art, as described in greater detail below, the scanner 18 produces controlled magnetic gradient fields in the presence of a main magnetic field. The scanner is capable of, then, producing pulsed radiofrequency (rf) signals that perturb gyromagnetic materials in the patient to produce magnetic resonance signals that are acquired by means of an rf antenna. The FUS transducer assembly 20 produces pulsed ultrasonic energy that is directed to the patient 24 during the MRgFUS procedure. The ultrasonic energy is modulated so as to focus the energy on a region within the patient to be ablated. The regions in which energy is focused will increase in temperature and retain heat to cause destruction of target tissues. The scanner 18 aids in directing this tissue destruction by producing images both prior to the procedure, for use as guides, and during the procedure, indicative of temperature changes of targeted tissue.

The MRgFUS system or suite 12 further includes a magnetic resonance control system, designated generally by reference numeral 26. The control system will typically include hardware, software and firmware designed to regulate the application of controlled gradient fields within scanner 18, as well as the application of rf pulses during an imaging sequence. The control system further allows for collection of MR signals returned from the subjects during such imaging sequences. In present implementations, system 26 also performs processing on these signals, such as by two-dimensional fast Fourier transforms to render images before and during procedures. The system may also produce the thermographic images, and use these thermographic images to estimate temperatures of tissues as described below.

In the illustrated embodiment, a monitor 28 and various input/output devices 30 may be coupled to the MR control system 26. These permit a technician or clinician 32 present at the location of the scanner 18 to interact with the MR system and control production of the images. As described below, these may also facilitate exchange of images with a surgeon controlling the MRgFUS procedure from the control station 14, and enabling or disabling phases of the procedure in cooperation with the surgeon.

The MRgFUS system or suite 12 further includes an MR/MRgFUS local surgical control workstation or host computer 34. The local surgical control workstation 34 is coupled to both the scanner/FUS station 16 and to the MR control system 26 by means of a local network 36. In general, the local surgical control workstation34 may perform various functions in conjunction with those performed by the control system 26. In certain applications, the local surgical control workstation34 may be integrated with the MR control system 26. Both systems, in conjunction, allow for positioning of the FUS transducer assembly 20 with respect to the patient, movement of the transducer assembly and/or the table 22 to appropriately locate the transducer with respect to the patient, control or production of images and the processing of the images, control the configuration and delivery of ultrasonic energy to the patient, and communication with the control station 14 via a network connection as represented generally by reference numeral 38.

In accordance with aspects of the present technique, as more fully described below, the remotely-controlled MRgFUS system 10 is designed to share control functions between the MRgFUS system or suite 12 and the remote surgical control workstation 14. That is, because the patient positioning, patient concerns and similar issues require attention for the procedure immediately where the patient is located, one or more clinicians 32 will be located at the MRgFUS system. However, the remote surgical control workstation 14 may be remote from this system. In certain embodiments, the remote surgical control workstation 14 may be in the same general geographic location as the system on which the energy is delivered to the patient, such as in the same hospital or institution. However, the control of processes for the MRgFUS procedure is separated between the systems. This allows for the remote surgical control workstation 14 to be located, where desired, at entirely remote locations, and allows for control of the procedure to be done by a specialist who cannot or for other reasons is not physically located where the procedure is to be performed on the patient.

In the illustrated embodiment, the remote surgical control workstation 14 includes a control computer 40 which may be loaded with software for receiving instructions and running an application for control of the MRgFUS system or suite 12. However, in certain embodiments, the control computer 40 may simply receive screens or views from the system 12, display the views on a monitor 42 and allow for interaction with the system via input/output devices as indicated at reference numeral 44. The systems may therefore, where desired, operate in accordance with conventional collaborative computing approaches. Inputs made by a specialist, typically a surgeon 46 or the surgeon's team will be received by computer 40 and transmitted to the system 12 where imaging sequences can be launched, images can be produced and the patient may be treated. That is, some of all of the applications used to generate images and deliver controlled FUS energy to the patient may run on the MRgFUS system, with the remote surgical control workstation 14 merely displaying screens based upon data received from the MRgFUS system, receiving inputs by the surgeon, and transmitting the inputs to the MRgFUS system. Alternatively some of the applications may be operative on the control computer 40, or applications may be shared between these systems.

In general, the remote surgical control workstation 14 and the local surgical control workstation 34 may be essentially redundant. That is, MRgFUS procedures may be conducted under the control of the local workstation or the remote workstation. Moreover, software applications that control application of focused ultrasound energy, and more generally for the control of the imaging and/or FUS procedure may be loaded and run on either workstation or both. As described below, in a presently contemplated embodiment, for example, the applications may run on the local surgical control workstation 34, with views only being sent to the remote surgical control workstation 14. Inputs (e.g., keystrokes or mouse clicks) on the remote surgical control workstation 14 would then be encoded and transmitted to the local surgical control workstation 34 and treated as inputs for interpretation and action by the application.

It should also be noted that the present scenario for remotely or locally controlling MRgFUS procedures may be useful in various situations. As noted above, these may include where a qualified surgeon is not locally available for the procedure. Moreover, the arrangement facilitates supervision of procedures by qualified persons located remotely, such as in conjunction and cooperation with a team controlling the FUS procedure locally. Similarly, the arrangement facilitates mentoring and teaching of medical professionals located either at the local workstation or the remote workstation, or both.

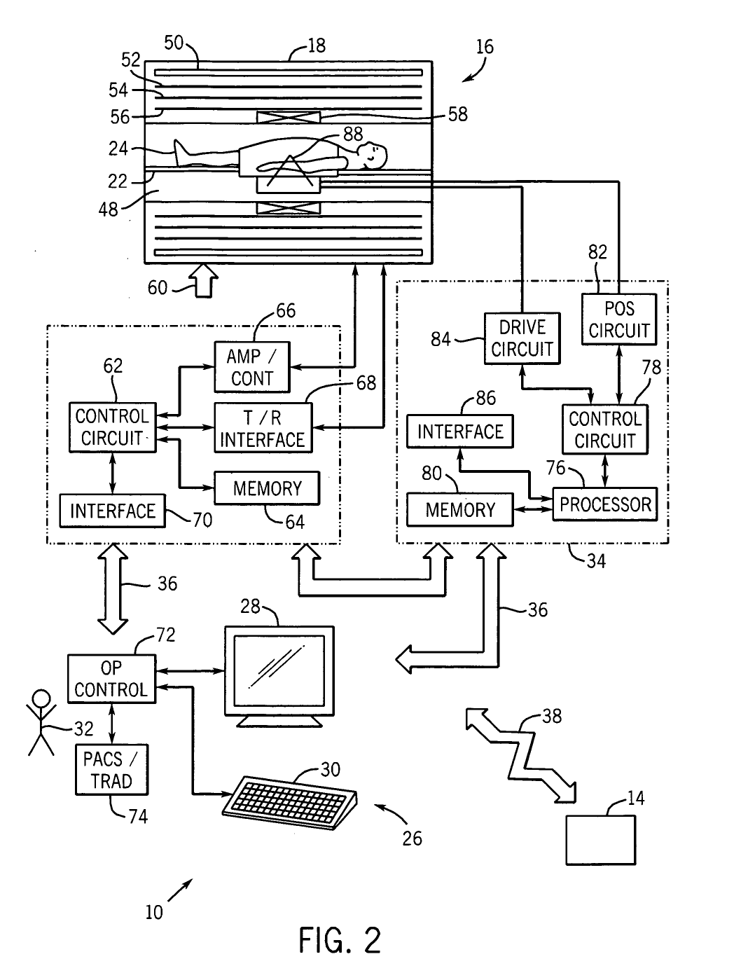

A somewhat more detailed diagrammatical representation of the MRgFUS system 12 is provided in FIG. 2. The system, as described above, includes a scanner/FUS station 16 that, itself, includes a magnetic resonance imaging scanner 18 and an FUS transducer assembly 20. The MRI scanner 18 may include any suitable MRI scanner or detector, and in the illustrated embodiment the system includes a full body scanner comprising a patient bore 48 into which a table 22 may be positioned to place a patient 24 in a desired position for scanning and for delivery of FUS energy. Scanner 18 further includes a series of associated coils for producing controlled magnetic fields, for generating rf excitation pulses, and for detecting emissions from gyromagnetic material within the patient in response to such pulses. In the diagrammatical view of FIG. 2, a primary magnet coil 50 is provided for generating a primary magnetic field generally aligned with patient bore 48. A series of gradient coils 52, 54 and 56 are grouped in a coil assembly for generating controlled magnetic gradient fields during examination sequences and MRgFUS procedures as described more fully below. A radiofrequency coil 58 is provided for generating rf pulses for exciting the gyromagnetic material. In the embodiment illustrated in FIG. 2, rf coil 58 also serves as a receiving coil. Thus, rf coil 58 may be coupled with driving and receiving circuitry in passive and active modes for receiving emissions from the gyromagnetic material and for applying rf excitation pulses, respectively. Alternatively, various configurations of receiving coils may be provided separate from rf coil 58. Such coils may include structures specifically adapted for target anatomies. Moreover, receiving coils may be provided in any suitable physical configuration, including phased array coils, and so forth.

In a present configuration, the gradient coils 52, 54 and 56 have different physical configurations adapted to their function in the imaging system. As will be appreciated by those skilled in the art, the coils are comprised of conductive wires, bars or plates which are wound or cut to form a coil structure which generates a gradient field upon application of controlled pulses as described below. The placement of the coils within the gradient coil assembly may be done in several different orders, but in the present embodiment, a Z-axis coil is positioned at an innermost location, and is formed generally as a solenoid-like structure which has relatively little impact on the rf magnetic field. Thus, in the illustrated embodiment, gradient coil 56 is the Z-axis solenoid coil, while coils 52 and 54 are Y-axis and X-axis coils respectively.

The coils of scanner 18 are controlled by external circuitry to generate desired fields and pulses, and to read signals from the gyromagnetic material in a controlled manner. As will be appreciated by those skilled in the art, when the material, typically bound in tissues of the patient, is subjected to the primary field, individual magnetic moments of the magnetic resonance-active nuclei in the tissue partially align with the field. While a net magnetic moment is produced in the direction of the polarizing field, the randomly oriented components of the moment in a perpendicular plane generally cancel one another. During an examination sequence, an rf frequency pulse is generated at or near the Larmor frequency of the material of interest, resulting in rotation of the net aligned moment to produce a net transverse magnetic moment. This transverse magnetic moment precesses around the main magnetic field direction, emitting rf (magnetic resonance) signals. For reconstruction of the desired images, these rf signals are detected by scanner 18 and processed. In the present context, the signals are used to produce one or more guide images and a series of thermographic images that are used to determine heating of target tissues during the MRgFUS procedure. Although the images are typically produced under the control or by the clinician 32 at the patient location, the images are sent to the control station 14 where the surgeon controls delivery of FUS energy to the patient.

Gradient coils 52, 54 and 56 serve to generate precisely controlled magnetic fields, the strength of which vary over a predefined field of view, typically with positive and negative polarity. When each coil is energized with known electric current, the resulting magnetic field gradient is superimposed over the primary field and produces a desirably linear variation in the Z-axis component of the magnetic field strength across the field of view. The field varies linearly in one direction, but is homogenous in the other two. The three coils have mutually orthogonal axes for the direction of their variation, enabling a linear field gradient to be imposed in an arbitrary direction with an appropriate combination of the three gradient coils.

The coils of scanner 18 are controlled by scanner control system 26 to generate the desired magnetic field and rf pulses. In the diagrammatical view of FIG. 1, control system 26 thus includes a control circuit 62 for commanding the pulse sequences employed during the examinations and MRgFUS procedures, and for processing received signals. For example, control circuit 62 applies analytical routines to the signals collected in response to the rf excitation pulses to reconstruct the desired guide images and thermographic data during the application of FUS energy, and computes temperature differences based upon the data. Control circuit 62 may include any suitable programmable logic device, such as a CPU or digital signal processor of a general purpose or application-specific determiner. Control system 26 further includes memory circuitry 64, such as volatile and non-volatile memory devices for storing physical and logical axis configuration parameters, examination pulse sequence descriptions, acquired image and thermographic data, programming routines, and so forth, used during the examination and treatment sequences implemented by scanner 18 and transducer assembly 20.

Interface between the control system 26 and the coils of scanner 18 is managed by amplification and control circuitry 66, and by transmission and receive interface circuitry 68. Circuitry 66 includes amplifiers for each gradient field coil to supply drive current to the field coils in response to control signals from control system 26. Interface circuitry 68 includes additional amplification circuitry for driving rf coil 58. Moreover, where the rf coil 58 serves both to emit the radiofrequency excitation pulses and to receive MR signals, circuitry 68 will typically include a switching device for toggling the rf coil 58 between active or transmitting mode, and passive or receiving mode. A power supply, denoted generally by reference numeral 60 in FIG. 2, is provided for energizing the primary magnet 50. Finally, circuitry 26 includes interface components 70 for exchanging configuration and image data, screen views, control signals and so forth with the other components of the workstation at which the imaging clinician 32 operates, as well as with the control station 14.

It should be noted that, while in the present description reference is made to a horizontal cylindrical bore imaging system employing a superconducting primary field magnet assembly, the present technique may be applied to various other configurations, such as scanners employing vertical fields generated by superconducting magnets, permanent magnets, electromagnets or combinations of these means. Additionally, while FIG. 2 illustrates a closed MRI system, the embodiments of the present invention are applicable in an open MRI system which is designed to allow access by a physician or clinician.

The workstation by which the on-site clinician 32 interacts with the MRgFUS system 12 may include a wide range of devices for facilitating interface between an operator or radiologist and scanner 18 (and transducer assembly 20) via control system 26. In the illustrated embodiment, for example, an operator controller 72 is provided in the form of a computer workstation employing a general purpose or application-specific computer. The station also typically includes memory circuitry for storing examination pulse sequence descriptions, examination protocols, user and patient data, image data, both raw and processed, and so forth. The station may further include various interface and peripheral drivers for receiving and exchanging data with local and remote devices. In the illustrated embodiment, such devices include a conventional determiner keyboard 30 and various other input devices, such as a mouse, or the like. A printer may be provided for generating hard copy output of documents and images reconstructed from the acquired data. A monitor 28 is provided for facilitating operator interface. In addition, the system may include various local and remote image access and examination control devices, represented generally by reference numeral 74 in FIG. 2. Such devices may include picture archiving and communication systems (PACS), teleradiology systems, and the like.

The MRgFUS controller 34 similarly includes circuits that allow for control of the FUS transducer assembly 20. In particular, the controller includes a processor 76 designed to produce controlled ultrasonic pulses that are appropriated modulated for delivering energy to the target tissues within the patient. The processor 76 may also facilitate exchanges of control and feedback data between the control system 26 and the remote control station 14. Control circuitry 78 coupled to the processor 76 allows for control of the pulsed energy applied to the transducer assembly. In particular, control circuitry 78 may interface with a position circuit 82 which aids in positioning the transducer assembly at an appropriate location for concentration of energy to target tissues while avoiding certain trajectories where intervening tissues may be located. Control circuitry 78 also provides signals to drive circuitry 84 for generating the pulsed ultrasonic energy. In particular, control signals from circuit 78 may cause the drive circuitry 84 to regulate the amplitude of the drive signals, control the intensity of ultrasonic energy delivered to the transducer, and may control the phase between each of the transducer elements. For example, by shifting the phase between transducer elements, a location or focal distance of a focal zone created by the ultrasonic energy produced by the transducer assembly may be adjusted. Moreover, the phase between transducer elements may be changed, as may a mode of operation of the transducer assembly. The size and shape of the focal zone created by the ultrasonic energy may also be controlled in the manner generally known in the art. The delivery of the ultrasonic energy, typically referred to in FUS procedures as “sonication”, will thus be regulated by the processor, control circuitry, and drive circuitry under the remote control of the control station 14. Routines for calculating the parameters of the drive signals and control of the sonication process will typically be stored in a memory device 80 of the controller 34. Finally, interface circuitry 86 will typically be provided for exchanging data with external circuits and devices, including the MR control system 26 and the remote control station 14.

As noted above, the present technique allows for positioning and care of a patient by a clinician or a medical team immediately at the patient location. The clinician operating at the same location, then, may control the imaging system for producing MR guide images and thermographic images. Control of the actual sonication process, however, is done by the surgeon operating at the control station 14 rather than immediately at the MRgFUS system 12. That is, rather than an integrated control, the present process allows the surgeon to be remotely located. It should be borne in mind, however, that the system described above could allow for MRgFUS procedures to be performed locally. That is, the system described above may permit regulation of the delivery of FUS energy to a patient under the control of a surgeon operating at the control system 26, where the surgeon can be co-located with the se systems. However, where such is not the case, the surgeon may control the procedure as described below.

In a presently contemplated embodiment, an application for transmitting screens or data for generation of such screens, typically having the appearance of conventional graphical user interface pages, may be provided on the control system 26, or the controller 34. In either case, the application that provides such screens may allow or disallow certain inputs until other inputs are received. For example, where a certified imaging technician is present at the imaging location, that technician may aid in positioning the patient, ensuring that the system is ready for acquiring images, and may oversee and initiate the acquisition of MR guide images and later thermographic images. Alternatively, the clinician may be limited in doing so until an input is received from the surgeon at the control station 14. Conversely, at a stage in the process where sonication is to be performed by the delivery of FUS energy from the transducer assembly, the screens provided at the control system may call for specific input by the surgeon at the control station 14. That is, certain screen regions or inputs may be blocked from the local clinician, requiring authorized input by the surgeon. Hybrid blocking and permission schemes may be envisaged, in which a clinician operating at the immediate vicinity of the patient acknowledges and enables certain functions depending upon the local conditions (e.g., patient and system readiness), which then enables the surgeon to proceed in configuring and initiating sonication. Techniques for controlling, blocking and enabling such remote inputs are described in U.S. patent application Ser. No. 10/681,730, filed on Oct. 8, 2003, by Muralidharan et al., entitled “METHOD AND APPARAUTS FOR SELECTIVELY BLOCKING REMOTE ACTION”; U.S. patent application Ser. No. 10/722,725, filed on Nov. 25, 2003, by Deaven et al., entitled “METHOD AND APPARATUS FOR REMOTE PROCESSING OF IMAGE DATA”; and U.S. patent application Ser. No. 10/723,087, filed on Nov. 25, 2003, by Livermore et al., entitled “METHOD AND SYSTEM FOR REMOTE OPERATION OF MEDICAL IMAGING SYSTEM”, all of which are incorporated herein by reference in their entirety.

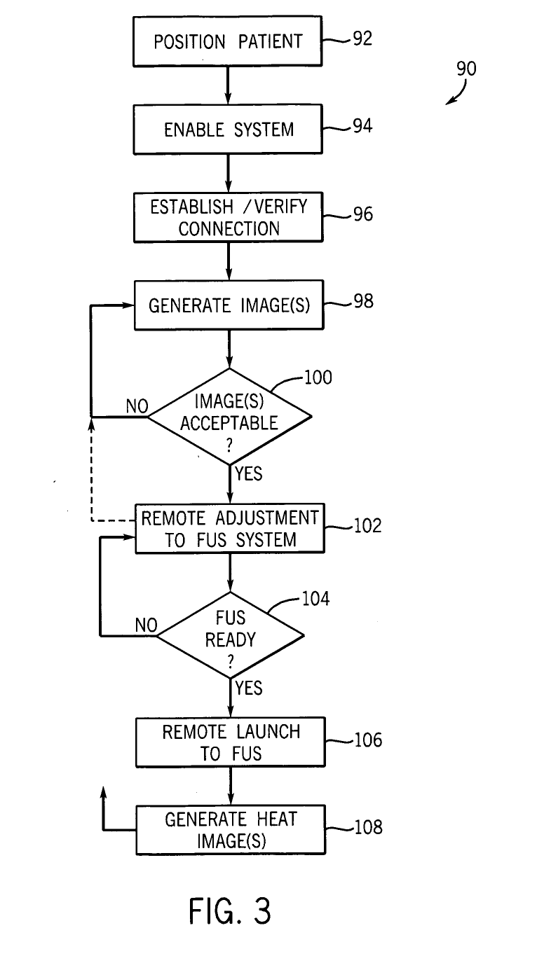

The remotely-controlled MRgFUS process will proceed through steps that are designed to verify the readiness for the process locally, as well as the control of the process remotely, as generally summarized in FIG. 3. Exemplary steps in the process, as indicated generally by reference numeral 90, begin at a step 92 where the patient is positioned in the scanner. In general, the patient will be positioned in the scanner based upon the tissues to be ablated. In uterine fibroid ablation procedures, for example, the patient will be positioned with her abdomen in close contact with the table, and a conductive gel or fluid ensuring good ultrasound transmission between the transducer assembly and the patient. Step 92 may include movement of the transducer assembly, typically along X and Y axes by means of position actuators associated with the transducer assembly.

At step 94, then, the system is enabled. Such enablement will typically include enablement of both the ultrasonic transducer assembly and the imaging system. In a typical process, the clinician operating at the patient location will see one or more graphical user interface screens that indicate system status, and these screens may be transmitted to the surgeon operating remotely. At step 96 the connection with the control station 14 will be established and verified. Where appropriate, special permissions and authentications may be required to permit the surgeon to enter into the system and proceed with authorizing processes to be performed remotely on the patient, as well as the processes to be input and commanded from the control station 14.

At step 98, one or more guide images will be generated. In general, these guide images will be high-resolution magnetic resonance images that show the tissues to be ablated and will serve the surgeon in orienting the focused ultrasound energy during the process. At step 100 the images can be indicated as acceptable by the surgeon. Again, the image will typically be made under the control of a clinician locally at the MRgFUS system and sent to the surgeon via the network link described above. Once acceptable guide images are obtained, the FUS system may be adjusted as indicated at step 102. As will be appreciated by those skilled in the art, such adjustment may be made to locate the tissues for ablation, position the transducer assembly, and so forth. Adjustments in the positioning may be made during the process of sonication, typically between periods of delivery of FUS energy. The system may be controlled by marks or indications made on the guide images by the surgeon operating at the control station 14. For example, such marks may indicate the location of tissues to be avoided in the delivery of the FUS energy, locations of skin lines, and so forth. Based upon such inputs by the surgeon, then, the sonication process may begin as indicated at step 104.

At step 104 the system may provide for verification by the local team or clinician that the patient is in position and the system is ready for sonication to begin. As noted above, this step may entail the local team enabling inputs by the surgeon, such as via regions displayed or “grayed” on a graphical user interface screen. If the system is not ready, the clinician may simply wait to enable the sonication process. Moreover, the clinician may interface with the surgeon to produce digital images, make further adjustments to the FUS system, and so forth.

As indicated at step 106, then, once the system is ready, the surgeon operating at the control station 14 may launch the FUS sonication steps. As will be appreciated by those skilled in the art, sonication is typically performed in a number of such steps, with thermographic images being produced during sonication, as indicated at step 108. The sonication periods themselves may last, for example, 20 to 30 seconds, with periods of cooling provided therebetween, such as on the order of 90 seconds. Such periods may, of course, vary depending upon such factors as the tissues to be ablated, the energy delivered, and particularly upon the temperatures and stored heat in the tissues as indicated by the thermographic images. As will be appreciated by those skilled in the art, the thermographic images will enable a temperature differences to be computed, providing an indication of both the temperature of the treated tissues, temperatures of surrounding tissues, and generally the heat retained by the tissues. Ablation or controlled destruction of the tissues will result from such heating. These images, as with the guide images, are provided to the surgeon at the control station 14, who in a similar manner launches or enables further sonication and imaging sequences. At any time during the process, the local clinician or team can disable the system and alert the surgeon to any changes that may require delay or other alteration of the surgical plan. For example, patient discomfort, patient movement, system irregularities, and so forth may be accommodated in this manner.

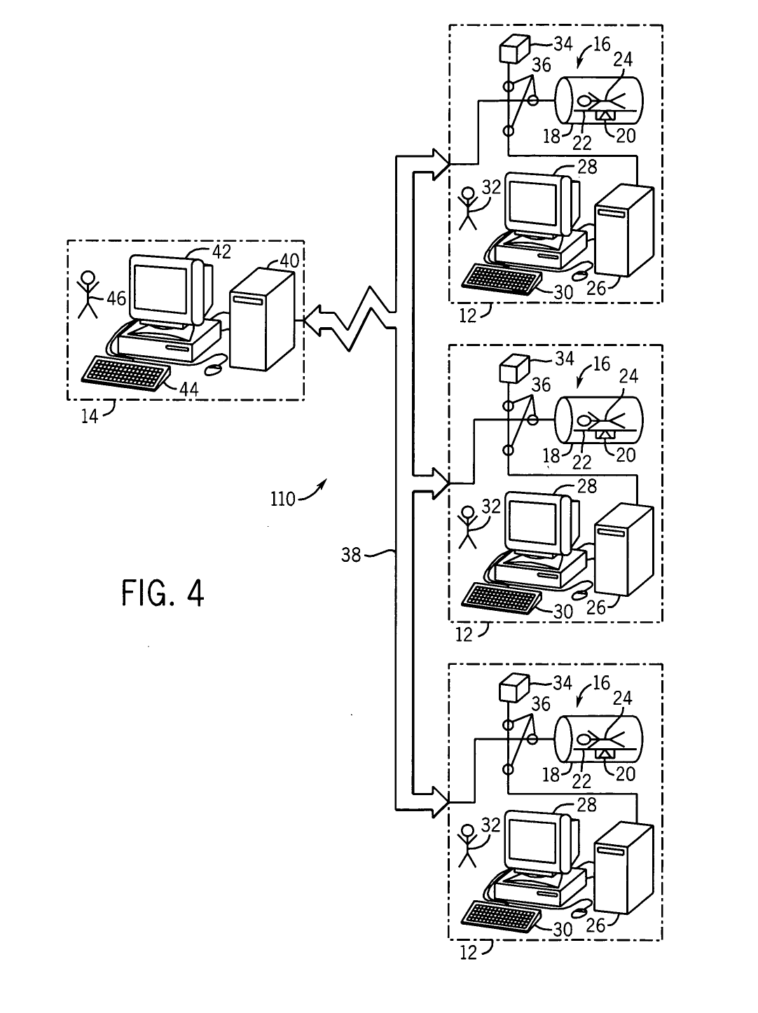

As noted above, the technique provided by the invention may allow for central control at a single control station, or by single or multiple surgeons, of MRgFUS procedures in many different locations. That is, a single surgeon or a single control station may serve as the base for procedures performed on MRgFUS systems at different locations. FIG. 4 generally illustrates a multi-system approach of this type. As shown in FIG. 4, the distributed system 110 will include a control station 14 of the type described above, but may be coupled to multiple MRgFUS systems 12 by means of a network 38. Each of the systems 12 may include components and functionalities similar to those described above. However, the systems may be at quite different locations, including locations around the world. The surgeon, then, interfaces successively with each system for performing the MRgFUS procedures. In a presently contemplated implementation, for example, the surgeon may schedule such procedures in coordination with remote teams in each of the locations and launch delivery of energy for ablation of patient tissues at regional locations based upon respective guide images and thermographic images acquire on individual systems.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for controlling a magnetic resonance guided focused ultrasound (MRgFUS) surgical procedure comprising:
establishing a network link between a remote surgical control workstation and a MRgFUS system, the MRgFUS system including a local surgical control workstation and being controllable either by interaction with either the remote surgical control workstation or the remote surgical control workstation;
generating a magnetic resonance guide image on the MRgFUS system;
transmitting the image to the surgical control workstation;
performing a focused ultrasound (FUS) ablation procedure at the MRgFUS system under the control of the remote surgical control workstation via the network link;
generating heat images on the MRgFUS system; and
transmitting the heat images to the remote surgical control workstation.

2. The method of claim 1, wherein the remote surgical control workstation is located in a different physical facility from the MSgFUS system.

3. The method of claim 1, wherein the network link includes the Internet.

4. The method of claim 1, wherein generation of the guide image is controlled locally at the MRgFUS system.

5. The method of claim 1, wherein generation of the heat images is controlled locally at the MRgFUS system.

6. The method of claim 1, comprising transmitting an electronic message from the MRgFUS system to the remote surgical control system indicating that the MRgFUS system is prepared for generation of the guide image or the heat images.

7. The method of claim 6, wherein the message is sent at the command of an operator at the MRgFUS system.

8. The method of claim 1, comprising transmitting an electronic message from the MRgFUS system to the remote surgical control system indicating that the MRgFUS system is prepared for the FUS ablation procedure.

9. The method of claim 8, wherein the message is sent at the command of an operator at the MRgFUS system.

10. A method for controlling magnetic resonance guided focused ultrasound (MRgFUS) surgical procedures comprising:
establishing network links between a surgical control workstation and a plurality of MRgFUS systems;
generating a magnetic resonance guide images on the MRgFUS systems;
transmitting the images to the surgical control workstation;
performing focused ultrasound (FUS) ablation procedures at the MRgFUS systems under the control of the surgical control workstation via the network links;
generating heat images on the MRgFUS systems; and
transmitting the heat images to the surgical control workstation.

11. The method of claim 10, wherein the surgical control workstation is located in a different physical facility from each of the MSgFUS systems.

12. The method of claim 10, wherein the network link includes the Internet.

13. The method of claim 10, wherein generation of the guide images is controlled locally at each of the MRgFUS systems.

14. The method of claim 10, wherein generation of the heat images is controlled locally at each of the MRgFUS systems.

15. A system for controlling a magnetic resonance guided focused ultrasound (MRgFUS) surgical procedure comprising:
a remote surgical control workstation;
a MRgFUS system, the MRgFUS system including a local surgical control workstation and being controllable either by interaction with either the remote surgical control workstation or the remote surgical control workstation;
a network link between the remote surgical control workstation and the MRgFUS system;
wherein the MRgFUS system is configured to generate a magnetic resonance guide image, to transmit the guide image to the remote surgical control workstation, and to perform a focused ultrasound (FUS) ablation procedure under the control of the remote surgical control workstation via the network link.

16. The system of claim 15, wherein the remote surgical control workstation is located in a different physical facility from the MSgFUS system.

17. The system of claim 15, wherein the network link includes the Internet.

18. The system of claim 15, wherein the MRgFUS system includes a local control workstation.

19. The system of claim 18, wherein the local control workstation is configured to control generation of the guide image.

20. The system of claim 19, wherein the local control workstation is configured to initiate acquisition of the guide image only upon a command received from the remote surgical control workstation.

21. The system of claim 15, wherein the MRgFUS system is configured to generate heat images during the FUS ablation procedure.

22. The system of claim 16, wherein the MRgFUS system is configured to transmit the heat images to the remote surgical control workstation.

23. The system of claim 15, wherein the MRgFUS system is configured to initiate the FUS procedure only upon a command received from the remote surgical control workstation.

24. The system of claim 23, wherein the MRgFUS system is configured to generate and transmit a message to the remote surgical control workstation indicating that the MRgFUS system is prepared for the FUS procedure.

25. The system of claim 15, wherein the remote surgical control workstation includes an application for controlling performance of the FUS procedure.
